(19) United States
(12) Patent Application Publication
(10) Pub. No.: US 2013/0137995 A1
(43) Pub. Date: May 30, 2013

(54) PULSE DIAGNOSIS DEVICE USING OPTICAL SENSOR

(71) Applicants: Seon Hoon Kim, Gwangju (KR); Doo-Gun Kim, Gwangju (KR); Hyun-Chul Ki, Gwangju (KR); Won-Gun Jang, Gwangju (KR); Dong-Kil Lee, Gwangju (KR); Hyo-Jin Kim, Gwangju (KR); Myung-Soo Han, Gwangju (KR); Hang-Ju Ko, Gwangju (KR); Hwe Jong Kim, Gwangju (KR); Byung-Teak Lee, Gwangju (KR)

(72) Inventors: Seon Hoon Kim, Gwangju (KR); Doo-Gun Kim, Gwangju (KR); Hyun-Chul Ki, Gwangju (KR); Won-Gun Jang, Gwangju (KR); Dong-Kil Lee, Gwangju (KR); Hyo-Jin Kim, Gwangju (KR); Myung-Soo Han, Gwangju (KR); Hang-Ju Ko, Gwangju (KR); Hwe Jong Kim, Gwangju (KR); Byung-Teak Lee, Gwangju (KR)

(73) Assignee: KOREA PHOTONICS TECHNOLOGY INSTITUTE, Gwangju (KR)

(21) Appl. No.: 13/813,634

(22) PCT Filed: Nov. 25, 2010
(86) PCT No.: PCT/KR10/08374

§ 371 Date: Jan. 31, 2013

(30) Foreign Application Priority Data
Aug. 6, 2010 (KR) 10-2010-0075826

(51) Int. Cl. A61B 5/024

(52) U.S. Cl. CPC A61B 5/02427
USPC 600/479

(57) ABSTRACT

A pulse diagnosis device which can detect the pulsation signal of a radial artery using an optical sensor comprising: a sensor module for sensing the pulsation signal by closely adhering thereto a prescribed body part; and a system control portion for operating the sensor module, and processing the optical signal sensed from the sensor module, wherein the sensor module comprises: an optical waveguide-type sensor which is placed on the bottom surface of the sensor module, and lets the optical signal to pass therethrough and detects the change in optical characteristics due to the change in the pressure; a light-source module which is connected on one side surface of the optical waveguide-type sensor, and inputs the optical signal into the optical waveguide-type sensor; and an optical detector module which is connected on one side surface of the optical waveguide-type sensor, and detects the optical signal delivered from the optical waveguide-type sensor.

### CROSS-REFERENCE TO RELATED APPLICATION(S)

This application claims the benefit of Korea Patent Application No. 10-2010-0075826, filed on Aug. 6, 2010, and in International Application No. PCT/KR2010/008374, filed on Nov. 25, 2010, titled “Pulse Diagnosis Device Using Optical Sensor,” the contents of which are hereby incorporated by reference in its entirety.

### BACKGROUND

1. Field

The present invention relates to a pulse diagnosis device using an optical sensor, and more specifically to a pulse diagnosis device using an optical waveguide-type sensor with optical signal detection material, in other words, material in which the optical characteristics change according to the pressure. A pulse diagnosis device adopting such a method can provide a more precise detector which is easy to form with multi-channels, and a more compact device compared to a conventional method using an electric signal.

2. Background

In general, pulse diagnosis in oriental medicine is diagnosing the state of human internal organs by measuring the patient's pulse wave while applying pressure after placing three fingers on a portion of the radial artery. Recently various measuring devices for measuring the pulse wave have been developed.

i) A sensor using electrostatic capacity variation used in a conventional Wheesoo (named by the inventor) type pulse diagnosis device and Thod pulse diagnosis device (Thod medicom, Korea) has low sensitivity and size limitations. Therefore, there is a difficulty in analyzing the nervation of 27 to 28 pulses precisely and variously. ii) A film type pressure sensor is of a small size but also shows a signal of applied force, so it is impossible to use in pulse diagnosis. iii) In addition, an infrared sensor cannot measure nor apply pressure in three portions of Chon, Kwan, and Chuck, which are places of the wrist touched by the forefinger, middle finger, and ring finger for sensing the pulsing of the lung, liver, and kidney, respectively. Therefore, it can only show the elasticity of the blood vessel through a simple flow of the blood stream. Accordingly, it has a problem that the measurement parameters thereof do not agree in measuring method compared to medical pulse diagnosis.

To attempt to solve such problems with conventional pulse diagnosis devices, a pulse diagnosis device using a piezoelectric element and another pulse diagnosis device using a hall element have appeared recently. The most commonly used method in the pulse diagnosis devices currently being put to practical use is a method of measuring the change of pressure by converting it into an electric signal using mainly a piezoelectric element.

However, because a pressure sensor that converts pressure changes into an electrical signal like this uses an extra electric wire, there is a structural difficulty in miniaturizing the sensor. Further, errors occur in measuring since it is affected by the self-heating of the electric wire. In addition, there still exists a structural problem that in order to realize multi-channel formation piezoelectric material of a bulk form should be formed in an array form and a large quantity of electric wire should be connected.

Therefore, there is demand for i) a pulse diagnosis device which minimizes measuring error by using an optical signal that is relatively more precise than the processing of an electrical signal, and ii) a pulse diagnosis device which can attain structural miniaturization by realizing a small optical waveguide-type sensor in an array form also for the case of multi-channel formation.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments, a pulse diagnosis device for detecting a pulsation signal of a radial artery using an optical sensor is provided, comprising: a sensor module for detecting a pulsation signal by closely adhering to a predetermined portion of a human body; and a system controller which drives the sensor module and processes an optical signal detected from the sensor module, wherein the sensor module comprises an optical waveguide-type sensor, which is positioned on the bottom surface of the sensor module, through which the optical signal passes, and which detects the change of optical characteristics according to the change of pressure; a light-source module which is connected to one side of the optical waveguide-type sensor and inputs the optical signal into the optical waveguide-type sensor; and an optical detector module which is connected to one side of the optical waveguide-type sensor and detects the optical signal delivered from the optical waveguide-type sensor.

These and other aspects of the claimed subject matter are described below.

### BRIEF DESCRIPTION OF DRAWINGS

The above objects, features and advantages of the present invention will become more apparent to those skilled in the related art in conjunction with the accompanying drawings.

FIG. 1 is a schematic view illustrating the appearance of a pulse diagnosis device according to an embodiment of the present invention;

FIGS. 2a and 2b are sectional views of the pulse diagnosis device according to an embodiment of the present invention;

FIG. 3 is an enlarged view of an optical waveguide-type sensor according to an embodiment of the present invention;

FIGS. 4a to 4c are sectional views illustrating various kinds of optical sensors used in the pulse diagnosis device according to an embodiment of the present invention;

FIG. 5 is a flowchart showing a method of detecting pulsation signals by using the pulse diagnosis device according to an embodiment of the present invention.

### DETAILED DESCRIPTION

In order to solve the problems with the method of detecting a pulse diagnosis signal in an electrical signal by using the above-described piezoelectric element, it is an object of the present invention to provide a precise pulse diagnosis device which minimizes measurement errors by detecting the change of pulse diagnosis signal in an optical signal form by using an optical waveguide-type sensor.

Another object of the present invention is to provide a pulse diagnosis device which can detect a pulse diagnosis signal on multi-channels by manufacturing a small optical waveguide-type sensor in a form of one or more arrays, and is relatively miniaturized compared to the method of realizing a multi-channel form by using a bulk piezoelectric element.

The technical tasks that the present invention is to accomplish are not limited by the technical tasks mentioned above. Any person who has common knowledge in the technical field to which the invention pertains can clearly understand other technical tasks not mentioned in the description of the present invention.

### TECHNICAL SOLUTION

In order to accomplish the foregoing objects, according to an aspect of the present invention, there is provided a pulse diagnosis device for detecting a pulsation signal of a radial artery using an optical sensor, including: a sensor module for detecting a pulsation signal by closely adhering to a predetermined portion of a human body; and a system controller which drives the sensor module and processes an optical signal detected from the sensor module, wherein the sensor module includes an optical waveguide-type sensor, which is positioned on the bottom surface of the sensor module, through which the optical signal passes, and which detects the change of optical characteristics according to the change of pressure; a light-source module which is connected to one side of the optical waveguide-type sensor and inputs the optical signal into the optical waveguide-type sensor; and an optical detector module which is connected to one side of the optical waveguide-type sensor and detects the optical signal delivered from the optical waveguide-type sensor.

Preferably, in the present invention, the optical waveguide-type sensor and the light-source module, and the optical waveguide-type sensor and the optical detector module are connected by optical fibers.

Preferably, in the present invention, the system controller includes a circuit module which drives the light-source module and the optical detector module, and processes the optical signal delivered from the optical detector module; and a connector which connects the light-source module and the optical detector module, and the circuit module, respectively.

Preferably, in the present invention, the sensor module includes one or more sensor modules formed in an array form.

Preferably, in the present invention, one or more pairs of optical fiber blocks for inputting or outputting an external optical signal are formed at opposite ends of the optical waveguide-type sensor.

Preferably, in the present invention, the optical waveguide-type sensor includes a main waveguide which includes a core and a cladding layer surrounding the core, and has an optical coupling area where the optical signal is branched; and a resonator which is arranged adjacent to the optical coupling area to receive a branched optical signal, and includes a piezoelectric material in a predetermined portion.

Preferably, in the present invention, the optical waveguide-type sensor is a pressure detecting optical sensor in which a predetermined portion of the cladding layer is etched, and a piezoelectric material is deposited in the etched portion.

Preferably, in the present invention, the piezoelectric material is formed in a thin film structure or an optical crystal structure.

Preferably, in the present invention, the piezoelectric material includes any one selected from zinc oxide (ZnO), aluminum nitride (AlN), cadmium sulfide (CdS) and piezoelectric zirconate titanate (PZT).

Preferably, in the present invention, the optical waveguide-type sensor includes one input end, one output end, and two or more optical channels, and is configured of a Mach-Zehnder electro-optic modulator type optical sensor in which the optical signal incident is branched one or more times.

According to another aspect of the present invention, there is provided a method for detecting a pulsation signal using a pulse diagnosis device provided with an optical sensor, including: adhering a sensor module to a predetermined portion of a human body;

inputting an optical signal into an optical waveguide-type sensor by driving a light-source module in a circuit module; detecting a pulsation signal from the optical waveguide-type sensor; detecting an optical signal from the optical waveguide-type sensor in which the pulsation signal is detected by an optical detector module; and processing the optical signal delivered from the optical detector module in the circuit module.

Preferably, in the present invention, the step of inputting the optical signal into the optical waveguide-type sensor and the step of detecting the optical signal from the optical waveguide-type sensor include inputting or detecting an optical signal through one or more pairs of optical fiber blocks formed at opposite ends of the optical waveguide-type sensor.

According to the present invention, there is provided a precise pulse diagnosis device which detects a pulse diagnosis signal by using optical signals and relatively minimizes the measurement error compared to the method of detecting which uses an electrical signal like a piezoelectric element. That is, it is possible to realize a precise sensor with high sensitivity through the piezoelectric thin film and the piezoelectric optical crystal structure provided in the optical waveguide-type sensor of the present invention. Thus, it is possible to minimize the measurement error of the pulse diagnosis device.

In addition, according to the present invention, it is possible to detect pulse diagnosis signals on multi-channels by manufacturing a small optical waveguide-type sensor in one or more array forms. Therefore, it is possible to provide a relatively miniaturized pulse diagnosis device compared to the method of realizing multi-channels by using a bulk piezoelectric element.

That is, according to the present invention, optical sensors of various kinds having piezoelectric material in a thin film or optical crystal structure are realized, so that compared to the sensor type in which the shape of electric polarization generated by mechanical modification is extracted, additional elements such as electric wire for drawing out electric signals to outside are not necessary. That is, using an optical sensor with high sensitivity, it is possible to manufacture a pulse diagnosis device which is structurally small, sensitive and precise.

### BEST MODE

Hereinafter, preferable embodiments of the present invention will be described with reference to the accompanying drawings.

Prior to this, terms or words used in the specification and claims should not be construed as limited to a lexical meaning, and should be understood as appropriate notions by the inventor based on that he/she is able to define terms to describe his/her invention in the best way to be seen by others. Therefore, embodiments and drawings described herein are simply exemplary and not exhaustive, and it will be understood that various modifications and equivalents may be made to take the place of the embodiments.

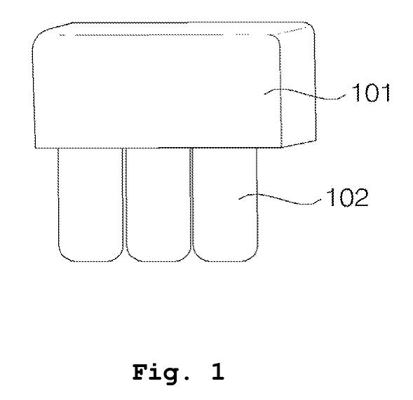

FIG. 1 is a schematic view illustrating the appearance of a pulse diagnosis device according to an embodiment of the present invention.

Compared to the conventional method using electrical signals, the present invention realizes a more precise detector and proposes a pulse diagnosis device which is easy to form with multi-channels and is miniaturized. Further, the present invention proposes a pulse diagnosis device which is more precise and minimizes measurement error by using an optical waveguide-type sensor provided with an optical signal detection material whose optical characteristics are changed by pressure.

FIG. 1 illustrates the appearance of a pulse diagnosis device using an optical sensor according to the present invention. The pulse diagnosis device can be divided largely into a housing 102 of a sensor module and a housing 101 having a system controller. Especially the sensor module can be formed with multi-channels by using an optical waveguide-type sensor, and can be made with a small size. Therefore, a plurality of detection modules can be provided in a limited area, so it has an advantage that the measurement of pulsation signals is more precise and easy.

Below will be described in detail the internal structure of a pulse diagnosis device using an optical sensor proposed in the present invention.

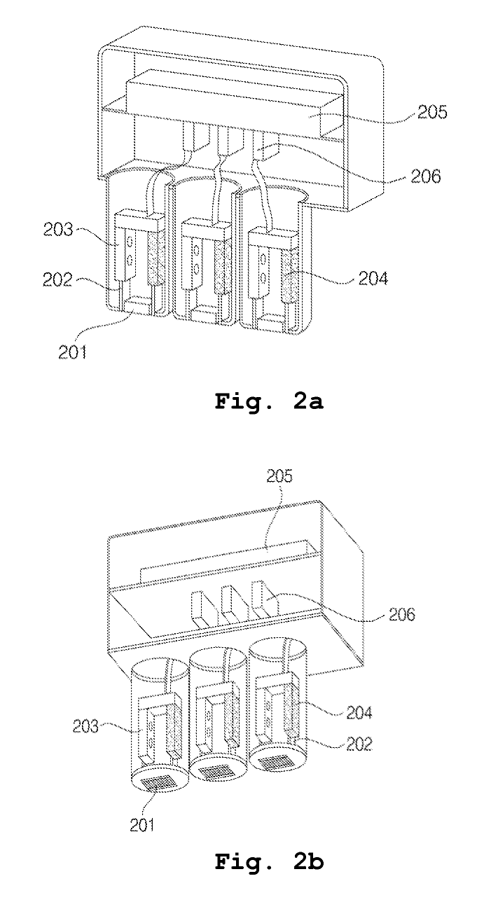

FIGS. 2a and 2b are sectional views of the pulse diagnosis device according to an embodiment of the present invention.

The pulse diagnosis device using the optical sensor according to the present invention detects pulsation signals of the radial artery using an optical sensor. This pulse diagnosis device can include a sensor module which is adhered to a predetermined portion of the human body for detecting pulsation signals, and a system controller which drives the sensor module and processes optical signals detected from the sensor module.

It is preferable that the sensor module include an optical waveguide-type sensor 201, a light-source module 203 and an optical detector module 204.

The optical waveguide-type sensor 201 is located on the bottom surface of the sensor module and optical signals pass therethrough. It plays a role of detecting the change of optical characteristics according to the change of pressure.

The optical waveguide-type sensor 201 is placed on the bottom surface of the sensor module. Therefore, when adhered or close to a predetermined portion of a human body, it detects pressure change due to pulsation signals by using piezoelectric material, or the like.

In particular, the optical waveguide-type sensor 201 may be configured by an optical sensor provided with piezoelectric material of a thin-film structure or an optical crystal structure. Further, it may also be configured by an optical sensor provided with a resonator having piezoelectric material, or a Mach-Zehnder electro-optic modulator type optical sensor. Since it is possible to include optical sensors of various types like this, it has an advantage that it is possible to provide optical waveguide-type sensors 201 of various types by considering the conditions such as the portions of the human body for which pulsation signals are to be measured.

The light-source module 203 is connected to one side of the optical waveguide-type sensor 201, and plays a role of inputting optical signals into the optical waveguide-type sensor 201. Of course, it is also possible to input or output optical signals by forming an optical fiber block at opposite ends of the optical waveguide-type sensor 201 as necessitated by the invention.

The optical detector module 204 is connected to one side of the optical waveguide-type sensor 201. At this time, it is preferable to connect to the other side to which the light-source module 203 is not connected. The optical detector module 204 plays the role of detecting the optical signals delivered from the optical waveguide-type sensor 201. That is, pressure change due to pulsation signal is detected as a change of optical characteristics of an optical signal by the optical waveguide-type sensor 201, and the detected signal is transmitted to the optical detector module 204.

The detected optical signal delivered from the optical waveguide-type sensor 201 is transmitted to a circuit module 205 of the system controller through the optical detector module 204, and the transmitted optical signal is processed in the circuit module 205.

The light-source module 203 of the present invention is not particularly limited, and any light-source module in the public domain may be used, if it can perform the function of inputting an optical signal to the optical sensor. Also, the optical detector module 204 is not particularly limited, and any optical detector module may be used, if it can receive and transfer the optical signal.

It is preferable that i) the optical waveguide-type sensor and the light-source module 203, and ii) the optical waveguide-type sensor and the optical detector module 204 be connected with optical fibers 202.

It is preferable that one or more sensor modules be formed in an array form. This is because the greater the number of sensor modules, the more precisely the pulsation signal can be measured. In the present invention, the sensor is configured by using the optical waveguide-type sensor 201 that includes piezoelectric material. Therefore, it is possible to miniaturize the area and structure of the sensor module. Thus, the present invention has an advantage in that it is easy to form multi-channels compared to a related art.

The system controller may include the circuit module 205 and a connector 206.

The circuit module 205 can play the role of i) driving the light-source module 203 and the optical detector module 204 of the sensor module, and ii) receiving and processing the optical signals delivered from the optical detector module 204

The connector 206 connects the light-source module 203 and the optical detector module 204 of the sensor module, and the circuit module 205 for controlling the light-source module 203 and the optical detector module 204, and plays the role of relaying the signals from the optical detector module 204 for signal processing.

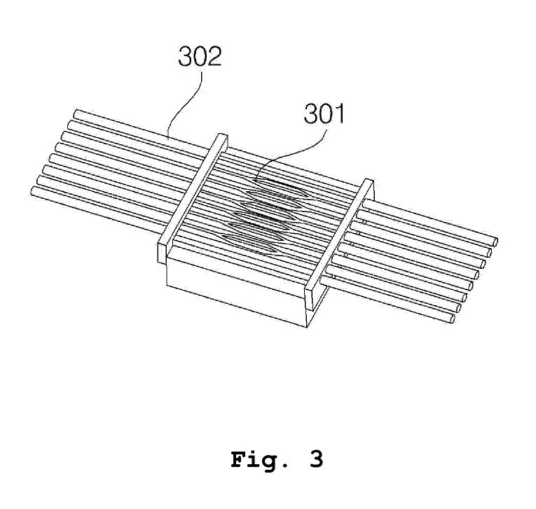

FIG. 3 is an enlarged view of an optical waveguide-type sensor according to an embodiment of the present invention.

The optical waveguide-type sensor 301 of the present invention may have one or more pairs of optical fiber blocks 302 arranged at opposite ends thereof for inputting or outputting external optical signals. Especially, if the sensor module is formed with multi-channels, it may have one or more pairs of optical fiber blocks 302 at each of opposite ends of a plurality of optical waveguide-type sensors 301.

Having optical fiber blocks 302 as above has an advantage that it is easy to transmit optical signals between the optical waveguide-type sensor 301 and the light-source module 203 and the optical detector module 204.

In the present invention, the optical waveguide-type sensor 301 can be configured by using various kinds of optical sensors.

For example, as optical sensors, there are i) a pressure detecting optical sensor in which a predetermined portion of a clad layer formed surrounding a core is etched and piezoelectric material of a thin film structure or optical crystal structure is deposited on the etched portion, ii) an optical sensor provided with a main waveguide which includes a core and a cladding layer surrounding the core, and has an optical coupling area where optical signals are branched, and a resonator which is arranged adjacent to the optical coupling area and receives branched optical signals and has piezoelectric material in a predetermined portion, iii) a Mach-Zehnder electro-optic modulator type optical sensor which is provided with one input end and one output end, includes two or more optical channels, and in which incident light signals are branched one or more times. It is possible to configure the optical waveguide-type sensor 301 by using such optical sensors.

The piezoelectric material may include any one selected from zinc oxide (ZnO), aluminum nitride (AlN), cadmium sulfide (CdS), and piezoelectric zirconate titanate (PZT).

The structure and operation of various kinds of optical sensors will be described below.

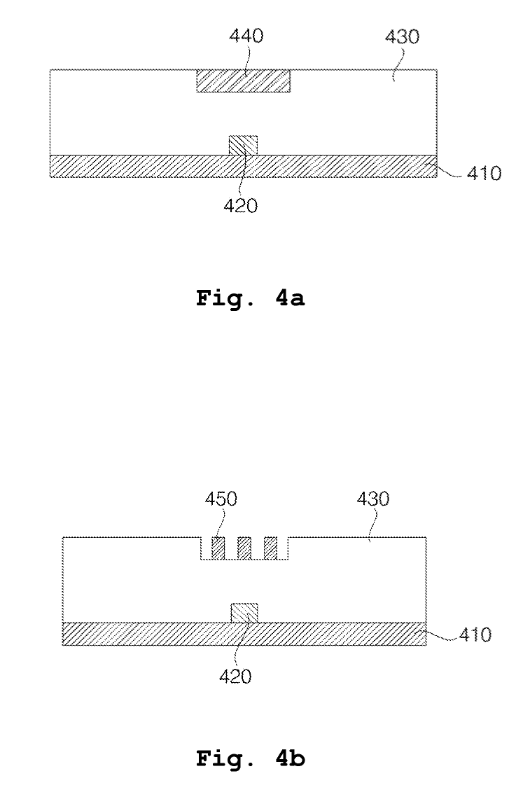

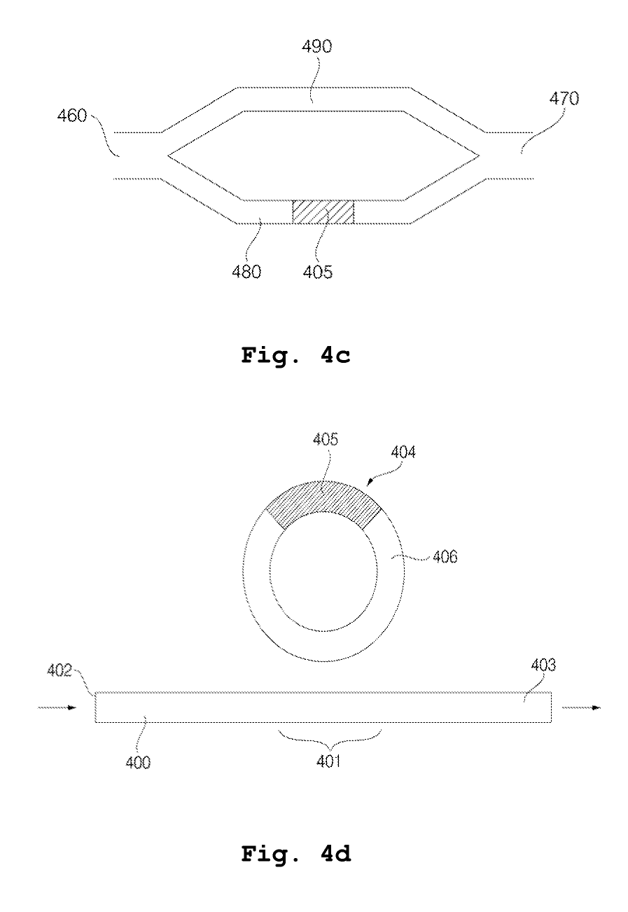

FIGS. 4a to 4c are schematic views illustrating various kinds of optical sensors used in the pulse diagnosis device according to an embodiment of the present invention.

FIG. 4a is a sectional view illustrating an optical sensor in which a predetermined portion of a clad layer surrounding a core is etched and the etched portion has piezoelectric thin film formed of piezoelectric material to detect pressure.

With reference to FIG. 4a, the optical sensor is formed of a piezoelectric body by etching a predetermined portion of a waveguide, which includes a lower clad layer 410 in which a core 420 having a refractive index higher than that of the clad layer is formed on the top surface and an upper clad layer 430 which is formed on the lower clad layer 410 so as to surround the core 420.

At this time, the lower clad layer 410 and the upper clad layer 430 are formed of the same material, and, although illustrated separately for the convenience of explanation, preferably they can be formed by inserting the core into one clad layer.

A method of manufacturing ‘the optical sensor with a piezoelectric body’ will be described briefly below.

First, a predetermined portion of the optical waveguide is etched in order to form a material that affects the light passing through the optical waveguide when pressure is applied, that is, a piezoelectric material that changes the refractive index of light. A piezoelectric thin film 440, which is the piezoelectric material, is formed in a predetermined portion of the etched upper clad layer 430.

The piezoelectric thin film 440 can be deposited in various ways such as a physical vapor deposition method, chemical vapor deposition method and liquid phase method. It is necessary to form a single crystal thin film of uniform thickness in order to show excellent piezoelectric characteristics. At this time, the thickness of the piezoelectric thin film is several hundred nanometers to several micrometers, and the thickness may be different according to the piezoelectric characteristics to be measured.

More specifically, the piezoelectric material is made by forming a buffer layer using the physical vapor deposition method or chemical vapor deposition method such as sputtering, molecular beam epitaxy (MBE), and metalorganic chemical vapor deposition (MOCVD), and growing thereon into a thin film form having good crystallinity by a liquid phase method.

The piezoelectric material is a material in which polarization is induced inside the material or mechanical deformation is caused by an external electric field when external mechanical pressure is applied. Though not particularly limited, the piezoelectric material of the present invention may include any one selected from zinc oxide (ZnO), aluminum nitride (AlN), cadmium sulfide and piezoelectric zirconate titanate (PZT), and preferably zinc oxide (ZnO).

In general, a sensor using a piezoelectric body makes use of electric polarization generated mainly by mechanical deformation, but there are also vibration, acceleration, angular velocity, and acoustic sensors. Another characteristic of the piezoelectric body is showing the change of refractive index by pressure and external stress.

In the present invention, a piezoelectric material (piezoelectric thin film or piezoelectric optical crystal) is formed on the optical waveguide to make use of the characteristic of showing the change of refractive index by pressure. Thus, if pressure is applied to the piezoelectric material, the refractive index of the piezoelectric material is changed, and this affects the characteristics of the light passing through the optical waveguide. Therefore, it is possible to detect the pressure applied from outside through the optical characteristics changed by the effect of the refractive index.

FIG. 4b is a sectional view showing an example of an optical sensor for detecting pressure applied to the optical waveguide-type sensor. This optical sensor is formed by etching a predetermined portion of the clad layer surrounding the core and depositing piezoelectric optical crystals on the etched portion.

A piezoelectric optical crystal 450 can be formed through the etching process or growth process, and the etching process is carried out using a nano-patterning process. The piezoelectric material of thin film form is deposited on the cladding layer 430 exposed by etching. By etching using electron beam lithography, nano-imprint and laser interference lithography, it is possible to form optical crystals with piezoelectric material of a thin film by using nano-patterns.

In addition, the growth process, after forming a buffer layer (not shown) in the cladding layer 430 exposed by etching, forms nano-patterns, and by using the patterns, can form the piezoelectric optical crystal 450 by selectively growing using the liquid-phase source of piezoelectric material.

Thus, by using the optical sensor having piezoelectric material of a thin film or optical crystal structure, it is possible to form an optical sensor which can be miniaturized compared to a bulk type (lump) piezoelectric sensor and which has improved sensitivity. If the piezoelectric material is formed of an optical crystal structure, the effective piezoelectric constant increases compared to a bulk type, so that it has an advantage of improved sensitivity of the optical sensor.

FIG. 4c is a view illustrating a Mach-Zehnder electro-optic modulator type sensor applied to an optical waveguide-type sensor.

FIG. 4c shows a Mach-Zehnder electro-optic modulator which includes one input end 460, one output end 470, and two optical channels (optical waveguides) 480 and 490 as the middle channel is branched. FIG. 4c illustrates optical channels 480 and 490 divided into two by the branching of one, but it is not limited thereto and the number of branching and the number of optical channels may be changed as necessitated by the invention.

It is preferable to form piezoelectric material 405 on one optical channel 480 of the two optical channels 480 and 490. At this time, the light incident from one input end 460 is branched and passes through each of the optical waveguides 480 and 490.

In addition, the light that passes through the optical waveguide 480 including the piezoelectric material 405 has a wavelength of light changed by the change of the refractive index of the piezoelectric material. The light having the changed wavelength is output after coupling with the light that has passed through another optical waveguide 490 in the portion of the output end 470.

Further, also the light that passes through the optical waveguide 490 in which the piezoelectric material 405 is not formed, can have the refractive index affected by the piezoelectric material 405 of the adjacent optical waveguide 480.

In the present invention, the piezoelectric material 405 can be formed of a piezoelectric thin film structure or a piezoelectric optical crystal structure.

At this time, the piezoelectric material 405 that is formed on the top or side of any one of the optical waveguides 480 and 490 reacts to external factors such as pressure, so the light that has passed through different optical waveguides shows a difference in the phase thereof. Accordingly, a difference occurs between the characteristics of the light measured at the output end 470 and the characteristics of the light incident from the input end 460, and it is possible to detect the pressure applied to the optical waveguide by the difference in the characteristics.

That is, the optical waveguide of a conventional optical crystal structure has the optical crystals disposed at an interval shorter than the wavelength of the light. By realizing a Mach-Zehnder electro-optic modulator type optical sensor through such an optical crystal structure, it is possible to measure the change of the effective refractive index of the optical waveguide with the minimum unit area.

Further, if the optical crystal is formed on the optical waveguide, only a specific wavelength passes; if another optical waveguide is positioned beside it, only a specific wavelength cannot pass. Such an optical characteristic of the specific wavelength has the resonance characteristic changed when the refractive index of the optical crystal is changed, so that the resonance wavelength moves or the phase is changed by the optical channel difference. That is, if the refractive index of the piezoelectric body of the optical crystal structure is changed, the resonance condition is changed, so that output becomes different or phase change occurs. Therefore, it is possible to measure the change of output optical wavelength by the phase change.

FIG. 4d is a view illustrating an optical sensor provided with a ring-type resonator applied to an optical waveguide-type sensor.

This optical sensor includes one main waveguide 400 through which light passes, and a resonator 404 arranged adjacent to the main waveguide 400. Though not shown in the drawing, the optical sensor may be configured by arranging the main waveguide 400 and the resonator 404 adjacent to each other on one substrate.

The main waveguide 400 is a conventional optical waveguide, and includes a cladding layer with a low refractive index and a core layer with a relatively high refractive index. The core layer is inserted into the cladding layer to transmit optical signals. In addition, opposite ends of the main waveguide 400 include an input end 402 at which optical signals are input and an output end 403 at which optical signals are output.

In the present invention, the resonator 404 can be configured in various shapes such as a ring shape, disk shape or polygon as necessitated by the invention.

The ring-type resonator 404 is formed of a conventional optical waveguide in the same way as the main waveguide 400, and may include a resonance waveguide 406 having a ring shape. Both the main waveguide and the resonance waveguide use the optical waveguide formed of the core layer and the cladding layer. Here, to make it easy to distinguish the two waveguides, the optical waveguide used in the resonator is named a resonance waveguide.

At this time, the portion to which the resonance waveguide 406 and the main waveguide 400 are adjacent becomes an optical coupling area 401. The optical signal that passes the main waveguide 400 is branched according to the resonance condition of the resonance waveguide, and the optical signal of the wavelength that meets the condition is transmitted to the resonance waveguide 406.

The resonance waveguide 406 includes a piezoelectric material 405 formed in a predetermined portion thereof. The piezoelectric material 405 formed at the farthest distance from the main waveguide 400 may include any one material of zinc oxide (ZnO), aluminum nitride (AlN), cadmium sulfide (CdS) and piezoelectric zirconate titanate (PZT), and preferably zinc oxide (ZnO).

In addition, the piezoelectric material 405 may be formed in a thin film or optical crystal structure by etching a predetermined portion of the cladding layer of the resonance waveguide 406 as necessitated by the invention.

The piezoelectric material 405 has a refractive index changed according to external conditions, such as pressure, to change the resonance conditions of the resonance waveguide 406. Therefore, the optical signal branched at the optical coupling area 401 and input into the resonator 404 is changed by the piezoelectric material, thus the optical signal output through the output end of the main waveguide 400 is changed.

Next, the operation of the optical sensor including the ring-type resonator will be described. The optical signal input through the input end 402 of the main waveguide 400 advances along the main waveguide 400 and is branched at the optical coupling area 401 of the resonator 404 located adjacent to the main waveguide 400, and the optical signal of the wavelength meeting the resonance condition of the resonator 404 is transmitted to the resonator 404.

The optical signal input into the resonator 404 is influenced by external factors such as pressure, which is a measured factor in the piezoelectric material 405 formed in a predetermined portion (whole or part of the top surface, or side surface). Therefore, the effective refractive index of the resonator 404 is also changed.

Further, the optical coupling conditions (the resonance conditions) from the main waveguide 400 to the resonator 404 are changed according to the changed effective refractive index of the resonator 404. At this time, the effective refractive index of the resonator 404 is changed corresponding to pressure or the like applied to the main waveguide 400. Since the signal (intensity, phase, etc.) of the light output through the output end 403 of the main waveguide 400 is varied, it is possible to detect the characteristics of the external factor such as pressure.

That is, in the optical sensor based on the ring resonator, when the input light is advanced along the optical waveguide (the main waveguide), only the optical signal having the wavelength that meets the resonance conditions of the ring resonator located beside the optical waveguide is coupled at the optical coupling area, and the optical signal having the wavelength not coupled advances along the output optical waveguide (the main waveguide).

In addition, the coupling resonance conditions vary with the phase change of the light in the optical waveguide (the resonance waveguide) in the ring resonator. Therefore, it is possible to obtain the output of the desired wavelength by making the optical phase change of the optical waveguide different. At this time, the resonance conditions are changed according to the change of the refractive index of the piezoelectric body formed on the top surface or side surface of the ring resonator, thus the change in the optical wavelength of the output light can be detected.

In addition, the optical signal having the changed wavelength is coupled with the optical signal that again passes through the optical waveguide (the main waveguide) at the optical coupling area, and it is possible to detect external factors such as pressure by measuring the characteristics of the optical signal output through the output end 403.

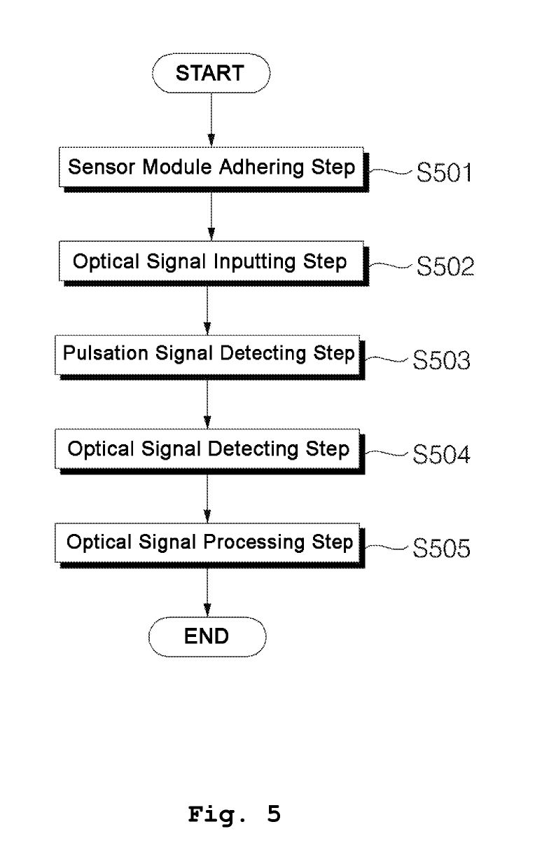

FIG. 5 is a flowchart showing a method of detecting pulsation signals by using the pulse diagnosis device according to an embodiment of the present invention.

First, a step in which the sensor module is adhered to a predetermined portion of the human body is carried out (S501).

In the present invention, since the sensor module is formed with multi-channels, pulsation signals can be measured more precisely.

Subsequently, by driving the light-source module in the circuit module, a step in which the optical signal is input in the waveguide type optical sensor is carried out (S502).

That is, when a driving signal is sent to the light-source module from the circuit module of the system controller, an optical signal is generated in the light-source module, and the optical signal is input into the optical waveguide-type sensor. Of course, it is also possible to input the optical signal through one or more pairs of optical fiber blocks formed at opposite ends of the optical waveguide-type sensor as necessitated by the invention.

Subsequently, the process goes through a step in which pulsation signals are detected from the optical waveguide-type sensor (S503).

The sensor module is adhered to a predetermined portion of the human body, and the pressure in the adhered portion is changed due to the pulsation signals of the human body. Through such a change of pressure, the change of optical characteristics is detected by using the piezoelectric material.

Subsequently, the process goes through a step in which the optical signals are detected by the optical detector module from the optical waveguide-type sensor that has detected pulsation signals (S504). Of course, it is also possible to input the optical signal through one or more pairs of optical fiber blocks formed at opposite ends of the optical waveguide-type sensor as necessitated by the invention.

Finally, by going through a step (S505) in which the optical signal delivered from the optical detector module is processed in the circuit module, measurement and analysis of pulsation signals become possible.

Although preferred embodiments of the present invention have been described in the above detailed description, the present invention is not restricted thereto. Therefore, those skilled in the art will appreciated that various variations and modification are possible in conventional production/research applications without departing from the scope and spirit of the present invention disclosed in the description, and such variations and modifications are dully within the appended claims.

### DESCRIPTION OF REFERENCE NUMERALS IN DRAWINGS

101: housing of system controller, 102: housing of sensor module
201, 301: optical waveguide-type sensor, 202: optical fiber
203: light-source module, 204: optical detector module
205: circuit module, 206: connector
302: optical fiber block, 400: main waveguide
401: optical coupling area, 402, 460: input end
403, 470: output end, 404: resonator
405: piezoelectric material, 406: resonance waveguide
410: lower clad layer, 420: core
430: upper clad layer, 440: piezoelectric thin film
450: piezoelectric optical crystal, 480, 490: optical waveguide

1. A pulse diagnosis device for detecting a pulsation signal of a radial artery using an optical sensor, comprising:
a sensor module for detecting a pulsation signal by closely adhering to a predetermined portion of a human body; and
a system controller which drives the sensor module and processes an optical signal detected from the sensor module,
wherein the sensor module comprises an optical waveguide-type sensor, which is positioned on the bottom surface of the sensor module, through which the optical signal passes, and which detects the change of optical characteristics according to the change of pressure;
a light-source module which is connected to one side of the optical waveguide-type sensor and inputs the optical signal into the optical waveguide-type sensor; and
an optical detector module which is connected to one side of the optical waveguide-type sensor and detects the optical signal delivered from the optical waveguide-type sensor.

2. The pulse diagnosis device according to claim 1, wherein the optical waveguide-type sensor and the light-source module, and the optical waveguide-type sensor and the optical detector module are connected by optical fibers.

3. The pulse diagnosis device according to claim 1, wherein the system controller comprises a circuit module which drives the light-source module and the optical detector module, and processes the optical signal delivered from the optical detector module; and
a connector which connects the light-source module and the optical detector module, and the circuit module, respectively.

4. The pulse diagnosis device according to claim 1, wherein the sensor module includes one or more sensor modules formed in an array form.

5. The pulse diagnosis device according to claim 1, wherein one or more pairs of optical fiber blocks for inputting or outputting an external optical signal are formed at opposite ends of the optical waveguide-type sensor.

6. The pulse diagnosis device according to claim 1, wherein the optical waveguide-type sensor comprises a main waveguide which includes a core and a cladding layer surrounding the core, and has an optical coupling area where the optical signal is branched; and
a resonator which is arranged adjacent to the optical coupling area to receive a branched optical signal, and includes a piezoelectric material in a predetermined portion.

7. The pulse diagnosis device according to claim 1, wherein the optical waveguide-type sensor is a pressure detecting optical sensor in which a predetermined portion of the cladding layer is etched, and a piezoelectric material is deposited in the etched portion.

8. The pulse diagnosis device according to claim 6, wherein the piezoelectric material is formed in a thin film structure or an optical crystal structure.

9. The pulse diagnosis device according to claim 6, wherein the piezoelectric material includes any one selected from zinc oxide (ZnO), aluminum nitride (AlN), cadmium sulfide (CdS) and piezoelectric zirconate titanate (PZT).

10. The pulse diagnosis device according to claim 1, wherein the optical waveguide-type sensor comprises one input end, one output end, and two or more optical channels, and is configured of a Mach-Zehnder electro-optic modulator type optical sensor in which the optical signal incident is branched one or more times.

11. A method for detecting a pulsation signal using a pulse diagnosis device provided with an optical sensor, comprising:
adhering a sensor module to a predetermined portion of a human body;
inputting an optical signal into an optical waveguide-type sensor by driving a light-source module in a circuit module;
detecting a pulsation signal from the optical waveguide-type sensor;
detecting an optical signal from the optical waveguide-type sensor in which the pulsation signal is detected by an optical detector module; and
processing the optical signal delivered from the optical detector module in the circuit module.

12. The method according to claim 11, wherein the step of inputting the optical signal into the optical waveguide-type sensor and the step of detecting the optical signal from the optical waveguide-type sensor comprise inputting or detecting an optical signal through one or more pairs of optical fiber blocks formed at opposite ends of the optical waveguide-type sensor.

13. The pulse diagnosis device according to claim 7, wherein the piezoelectric material is formed in a thin film structure or an optical crystal structure.

14. The pulse diagnosis device according to claim 7, wherein the piezoelectric material includes any one selected from zinc oxide (ZnO), aluminum nitride (AlN), cadmium sulfide (CdS) and piezoelectric zirconate titanate (PZT).
